(19) United States
(12) Patent Application Publication
(10) Pub. No.: US 2007/0292904 A1
(43) Pub. Date: Dec. 20, 2007

(54) Ephrin and EPH receptor mediated immune modulation

(75) Inventors: Chaim M. Roifman, North York (CA); Andrew Freywald, Thornhill (CA); Nigel Sharfe, Toronto (CA); Thomas Grunberger, Toronto (CA); Eyal Grunebaum, Toronto (CA)

Correspondence Address: JAGTIANI + GUTTAG, 10363-A DEMOCRACY LANE, FAIRFAX, VA, 22030, US

(73) Assignee: The Hospital for Sick Children, Toronto (CA)

(21) Appl. No.: 11/703,751

(22) Filed: Feb. 8, 2007

Related U.S. Application Data
(62) Division of application No. 10/482,771, filed on Jun. 29, 2004.
(60) Provisional application No. 60/302,385, filed on Jul. 3, 2001.

(51) Int. Cl. G01N 33/00

(52) U.S. Cl. 435/7.92

(57) ABSTRACT

Methods and compositions for immune modulation are described. The methods involve modulating an ephrin or Eph receptor which consequently modulates an immune response, in particular a T cell response, modulates immune adhesion cell, modulates chemotaxis and/or migration or modulates apoptosis. The method is useful in treating a variety of conditions, including autoimmune disease, allergy, graft versus host disease, transplant rejection and cancer.

### CROSS-REFERENCE TO RELATED APPLICATIONS

This application claims the benefit of U.S. provisional application Ser. No. 60/302,385, filed Jul. 3, 2001, which is incorporated by reference herein in its entirety.

### FIELD OF THE INVENTION

The invention relates to method and compositions for modulating an immune response.

### BACKGROUND OF THE INVENTION

Ephrins (Eph family receptor interacting), are ligands for the Eph receptors, which form the largest known family of receptor class tyrosine kinases (Zhou et al 1998). Currently eight ephrins are known. The ephrins are all membrane anchored proteins, either by glycosylphosphatidylinositol (GPI) (ephrinA1-A5), or a trans-membrane domain (ephrinB1-83). The Eph receptors are divided into two groups based upon their ligand binding Characteristics, EphA or EphB, according to the class of ephrin bound; although receptor-ligand specificity is generally considered to be degenerate within a group (Zhou et al 1998, Zisch and Pasquale 1997). It is a characteristic of the Eph receptor family that their ligands must be membrane bound or oligomerized in order to be active. Soluble monomeric forms of ephrins can inhibit Eph receptor signaling, although dimerized or oligomerized soluble forms can stimulate receptor autophosphorylation and signaling (Davis et al., 1994: Sakano et al. 1996). Ephrins and Eph receptors are typically most highly expressed in neural and endothelial cells and most descriptions of their function concern development of the nervous system and angiogenesis (Adams et al., 1999; Ciossek et al., 1998; Daniel et al., 1996; Drescher et al., 1995; Gao et al., 1999; Hornberger et al., 1999; O'Leary and Wilkinson, 1999, Pandey et al., 1995, Wang et al., 1998).

### SUMMARY OF THE INVENTION

In accordance with one aspect of the present invention, it is demonstrated that Eph receptors, such as EphB1, EphB2 and EphB6, are expressed in the T cell lineage, such as thymocytes, mature T cells and transformed T cell lines. The recognition of Eph receptor expression in the T cell lineage suggests that ephrins could be regulators of immune behavior such as T cell behavior and control a variety of T cell responses, including responses.

Accordingly, the present invention provides a method of modulating an immune response comprising administering an effective amount of a substance that modulates an ephrin or an Eph receptor to a cell or animal in need thereof.

In one embodiment, the invention provides a method of modulating a T cell response comprising administering an effective amount of a substance that modulates an ephrin or an Eph receptor to a cell or animal in need thereof.

The inventors have shown that treatment of T cells with a ligand for an Eph receptor induces the formation of cell-cell contact.

Accordingly, in another embodiment, the present invention provides a method of modulating immune cell adhesion comprising administering an effective amount of a substance that modulates an ephrin or an Eph receptor to a cell or animal in need thereof. In a preferred embodiment, the method modulates T cell adhesion.

In a further embodiment, the present invention provides a method of modulating chemotaxis and/or migration comprising administering an effective amount of a substance that modulates an ephrin or an Eph receptor to a cell or animal in need thereof.

In accordance with an aspect of the present invention, it is shown that ephrin-Eph receptor signaling modulates TCR/CD3 induced apoptosis in thymocytes.

Accordingly, the present invention also provides a method of modulating apoptosis comprising administering an effective amount of a substance that modulates an ephrin or an Eph receptor to a cell or animal in need thereof.

The prevent invention also includes pharmaceutical compositions comprising an effective amount of a substance that modulates an ephrin or an Eph receptor for use in modulating an immune response, preferably a T cell response, or modulating immune cell adhesion, preferably T cell adhesion, or modulating chemotaxis and/or migration as well as in modulating apoptosis.

Methods of modulating the immune response, in particular the T cell response, have applications in a wide variety of diseases, including cancer, autoimmune disease, allergy, graft versus host disease, and transplantation. Methods of modulating immune cell adhesion, chemotaxis and/or migration have applications in many diseases such as cancer.

In another aspect the invention provides a method for treating cancer by modulating an adhesive, migratory or chemotactic property of a cancer cell, comprising administering to a cancer cell or to an animal having cancer an effective amount of a substance that modulates an ephrin or an Eph receptor.

In another aspect, the present invention provides a pharmaceutical composition for modulating an immune response, modulating immune cell adhesion, modulating apoptosis, modulating cell proliferation, modulating chemotaxis or modulating immune cell migration, said composition comprising an effective amount of a substance that modulates an ephrin or an Eph receptor. Preferably, the substance is a substance selected from the group consisting of oligomeric or monomeric soluble ephrins. Eph receptors, antibodies capable of binding an ephrin or an Eph receptor, antisense molecules complementary to a nucleic acid molecule encoding an ephrin or an Eph receptor, peptide mimetics based on ephrins or Eph receptors, and non-proteinaceous compounds capable of binding to and activating or inhibiting an ephrin or an Eph receptor.

In another aspect, the present invention provides a kit comprising a pharmaceutical composition as described above, and instructions for use of the composition for modulating an immune response, modulating immune cell adhesion, modulating apoptosis, modulating cell proliferation, modulating chemotaxis or modulating immune cell migration.

In another aspect, the present invention provides a method for identifying a substance that modulates immune cell adhesion, comprising:

contacting an ephrin or an Eph receptor with a test substance; and

determining whether said ephrin or Eph receptor is modulated in the presence of said test substance, a modulation of said ephrin or Eph receptor being an indication that said test substance is useful for modulating immune cell adhesion.

In another aspect, the present invention provides a method for identifying a substance that modulates chemotaxis or immune cell migration, comprising:

contacting an ephrin or an Eph receptor with a test substance; and

determining whether said ephrin or Eph receptor is modulated in the presence of said test substance, a modulation of said ephrin or Eph receptor being an indication that said test substance is useful for modulating chemotaxis or immune cell migration.

In another aspect, the present invention provides a method for identifying a substance that modulates apoptosis, comprising:

contacting an ephrin or an Eph receptor with a test substance; and

determining whether said ephrin or Eph receptor is modulated in the presence of said test substance, a modulation of said ephrin or Eph receptor being an indication that said test substance is useful for modulating apoptosis.

In another aspect, the present invention provides a method for identifying a substance that modulates an immune response, comprising:

contacting an ephrin or an Eph receptor with a test substance; and

determining whether said ephrin or Eph receptor is modulated in the presence of said test substance, a modulation of said ephrin or Eph receptor being an indication that said test substance is useful for modulating an immune response.

In another aspect, the present invention provides a method for identifying a substance useful for treating cancer through modulation of an adhesive, migratory or chemotactic property of a cancer cell, comprising:

contacting an ephrin or an Eph receptor with a test substance; and

determining whether said ephrin or Eph receptor is modulated in the presence of said test substance, a modulation of said ephrin or Eph receptor being an indication that said test substance is useful for treating cancer through modulation of an adhesive, migratory or chemotactic property of a cancer cell.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The invention will now be described in relation to the drawings in which:

FIG. 1. Eph Receptor Expression in Human Thymocytes and T cells. Expression of the EphA2, EphB1, EphB2 and EphB6 receptors was examined by RT-PCR in human thymocytes, peripheral blood T-lymphocytes and the mature T cell line Jurkat. Control β-actin primers were included in each reaction. The expected product sizes are: β-actin—660 bp, EphA2—279 bp, EphB1—309 bp, EphB2—375 bp, EphB6—294 bp. The identity of the PCR products was confirmed by sequencing. Water controls (no DNA) were negative (not shown). A 100 bp size ladder is shown on the right (Gibco, BRL).

FIG. 2. EphA receptor expression in thymocytes and T cells. Expression of the EphA receptors in murine thymocytes and spleen T cells and in the human T cell leukemia line Jurkat was examined by RT-PCR. Positive lanes are marked with an asterisk. Water controls were negative (not shown). A 100 bp size ladder is shown on the left.

FIGS. 3A and 3B. Ephrin-B1 treatment of human T lymphocytes induces cell-cell adhesion. A. Human peripheral T cells were stimulated with 2.5 μg/ml of ephrin-B1 for 3 hours at 37° C. Formation of cell clusters was followed by inverted light microscopy at ×100 fold magnification. B. Jurkat T cells were stimulated with 2.5 μg/ml or ephrin-B1 for 1 hour at 37° C. and examined as in (A).

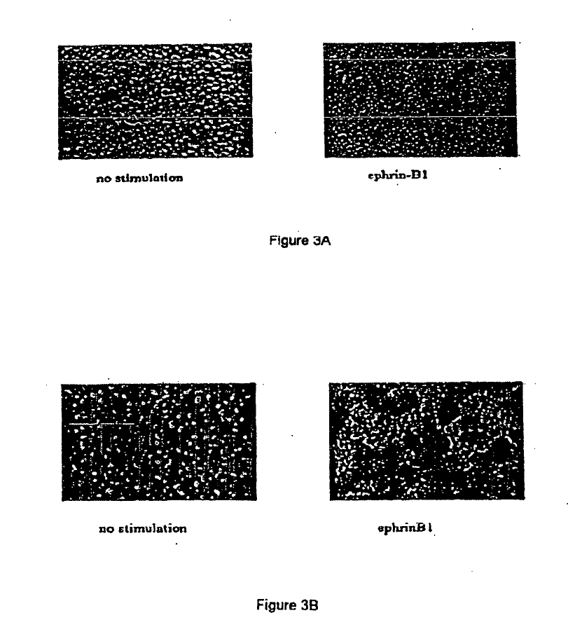

FIG. 4. Ephrin-B1-induced T cell adhesion is mediated by the LFA-1 integrin receptor. Anti-LFA-1 blocking antibody inhibits ephrin-B1-induced T cell adhesion. Peripheral T lymphocytes and Jurkat cells were stimulated with ephrin-B1 as in (FIG. 1) or with ephrin-B1 in the presence of 10 μg/ml of blocking anti-CD11a (a chain of LFA-1) T cell adhesion was followed as in (FIG. 3).

FIG. 5. Stable expression of Eph receptors in Jurkat T cells. The Jurkat T cell line was transfected with empty pcDNA3 (pcDNA3). EphB1-T7 (T7-tagged EphB1, B1-J), EphB6-M (Myc-tagged EphB6; B6-J) or DN (dominant negative)-EphB6 (Myc-tagged EphB6 with the intracellular domain deleted, DN-J). After 30 days of Geneticin selection, the expression of the transfected proteins was confirmed by immunoprecipitation with anti-T7 or anti-Myc and western plotting with the antibodies to the appropriate tag sequence as indicated.

FIG. 6. The EphB1 receptor is responsible for ephrin-B1 T cell adhesion. Overexpression of EphB1, but not EphB6 enhances T cell adhesion. Control pcDNA3 cells and B1-J, B6-J or DN-J cells were stimulated with 2.5 μg/ml of ephrin-B1, or ephrin-B1 in the presence of 10 μg/ml of blocking anti-CD11a (LFA-1 a chain), or in the presence of 10 μg/ml of non-blocking anti-CD18 (LFA-1 β chain) and analyzed as in FIG. 1B.

FIG. 7. Ephrin-B1 and ephrin-A1 inhibit T lymphocyte proliferation induced by stimulation through the T cell receptor complex. Cells were stimulated with immobilized anti-CD3 with or without ephrin-B1 or -A1 for 24 or 48 hours. Induction of DNA synthesis was analyzed by measuring the incorporation of 3H-thymidine.

FIG. 8. Ephrin-B1 and ephrin-A1 inhibit thymocyte apoptosis induced by stimulation through the T cell receptor complex. Cells were stimulated with immobilized anti-CD3 with or without ephrin-B1 or -A1 for 24 hours. Induction of apoptosis was analysed by annexin-V binding to cells using a FITC-conjugated annexin and flow cytometry.

FIG. 9. Ephrins modify chemotaxis toward SDF-1 alpha. Ephrin-Fc fusion proteins were immobilized on 5 μM Transwell membranes at 5 μg/ml. Membranes were then blocked with 5% milk and washed. Jurkat cells in serum free medium were loaded into the top chamber of the Transwell plate and 10 ng/ml human SDF-1α in the bottom. The plates were incubated at 37° C. for 2 hours and a sample of the cells that had migrated through the membrane into the bottom chamber counted on a flow cytometer.

FIG. 10. Ephrins modify the chemotaxis of human thymocytes toward the chemokine SDF-1 alpha. Ephrin-Fc proteins were immobilized on 3 μM or 5 μM Transwell membranes at 5 μg/ml, washed and blocked with 5% milk. Irrelevant Fc-fusion protein or purified human IgG were used as specificity controls. Thymocytes in medium containing 1% heat inactivated bovine serum were added to the top chamber and 10-100 ng/ml of SDF-1 alpha in the bottom. Plates were incubated at 37° C., 5% CO₂ for two hours and a sample of the cells that had migrated through the membrane into the bottom chamber wore counted. Representative assays with thymocytes from four individuals are shown.

FIG. 11. Ephrins modify the chemotaxis of human peripheral blood T lymphocytes toward the chemokine SDF-1 alpha. Ephrin-Fc proteins were immobilized on 3 μM or 5 μM Transwell membranes at 5 μg/ml, washed and blocked with 5% milk. Irrelevant Fc-fusion protein or purified human IgG were used as specificity controls. Purified T lymphocytes in medium containing 1% heat inactivated bovine serum were added to the top chamber and 10-100 ng/ml of SDF-1 alpha in the bottom. Plates were incubated at 37° C., 5% CO₂ for two hours and a sample of the cells that had migrated through the membrane into the bottom chamber were counted. Representative assays with T cells from two unrelated individuals are shown.

FIG. 12. Alteration of Eph receptor expression and/or function in T leukemia cells can alter their aggressiveness and tissue targeting in vivo. Jurkat leukemia T cells were stably transfected with mutants of the EphB6 or EphB1 receptors. These cells or the original unmodified cells were injected into immunodeficient mice (NOD-SCID). When animals demonstrated signs of sickness they were sacrificed and infiltration of leukemia cells into tissues analysed by staining of tissue sections and flow cytometry analysis of single cell suspensions of organs. Antibodies recognising human CD3 were used to identify the human cells—they are also morphologically distinct from mouse cells in tissue sections. EphB1**=mutated EphB1 receptor with null or partially interfering function, EphB6 DN=dominant negative EphB6 with its cytoplasmic domain deleted, pcDNA3=vector only transfected, i.e. control, N/A=not available, −=negative, +=detectable infiltration, +++=heavily infiltrated. Experiments are divided by horizontal lines. Both experiments 2 and 3 are ongoing and control mice remain alive and healthy (exp 2=approx day 140, exp 3=approx day 45).

### DETAILED DESCRIPTION OF THE INVENTION

I. Definitions

The term “animal” as used herein includes all members of the animal kingdom, preferably human.

The term “effective amount” as used herein means an amount effective, at dosages and for period of time necessary to achieve the desired result. For example, when the desired result is immune modulation, an effective amount is an amount effective to modulate an immune response. Whether an immune response has been modulated can be assessed by a number of in vivo or in vitro assays well known to those skilled in the art including, but not limited to, antibody assays (for example ELISA assay), antigen specific cytotoxicity assays, the production of cytokines or by observing the effect on a particular condition or disease.

The term “ephrin” as used herein means an Eph family receptor interacting protein that is a ligand for an Eph receptor. The term includes, but is not limited to, ephrin A1, ephrin A2, ephrin A3, ephrin A4, ephrin A5, ephrin A6, ephrin B1, ephrin B2 and ephrin B3.

The term “EPh receptor” as used herein includes all members of the Eph receptor family including, but not limited to EphA1, EphA2, EphA3, EphA4, EphA5, EphA6, EphA7, EphA8, EphB1, EphB2, EphB3, EphB4, EphB5, EphB6. The term includes one or more Eph receptor.

The term “immune cell” as used herein means any cell derived from a hematopoietic stem cell and includes, but is not limited to, T cells, B cells, NK cells, monocytes, macrophages, dendritic cells, thymocytes and progenitors of any of those (such as pre-I cells).

The term “immune response” as used herein means any response of the immune system, for example, of either a cell-mediated or a humoral nature.

The term “modulate” as used herein includes the inhibition or suppression of a physiological response as well as the induction or enhancement of a physiological response.

The term “modulating apoptosis” as used herein means that the substance evokes a change in the apoptosis of a cell and includes an increase or enhancement of apoptosis as well as a decrease or inhibition of apoptosis.

The term “modulating immune cell adhesion” as used herein means that the substance evokes a change in the adhesion or cell:cell contact or cell:matrix contact of an immune cell with another cell or matrix. The term includes an increase or enhancement of adhesion as well as a decrease or inhibition of adhesion. The cell that adheres to the immune cell can be any cell that can functionally associate with immune cells.

The term “modulating T cell adhesion” as used herein means that the substance evokes a change in the adhesion or contact between a T cell and its target such as a cell or matrix. The term includes an increase or enhancement of T cell adhesion as well as a decrease or inhibition of T cell adhesion. The target cell that adheres to the T cell can be any cell that can functionally associate with T cells including, but not limited to, any antigen presenting cell, epithelial cells in central and peripheral lymph tissue, bone marrow, gut and skin.

The term “modulating a T cell response” as used herein means that the substance evokes a chance in a T cell response and includes an increase or enhancement of the T cell response as well as a decrease or suppression in the T cell response.

The term “modulating an immune response” as used herein means that the substance evokes a change in an immune response and includes an increase or enhancement of the immune response as well as a decrease or suppression in the immune response.

The term “substance that modulates an ephrin or an Eph receptor” means that the substance interacts with an ephrin or an Eph receptor to result in a modulation in a physiological response. A modulation in a physiological response includes the modulation of an immune response, the modulation of a T cell response, the modulation of cell adhesion, the modulation of T cell adhesion, modulation of chemotaxis and/or migration, and the modulation of apoptosis and/or proliferation of cells. The “substance” includes both activators and inhibitors of an ephrin or Eph receptor.

II. Methods of Immune Modulation

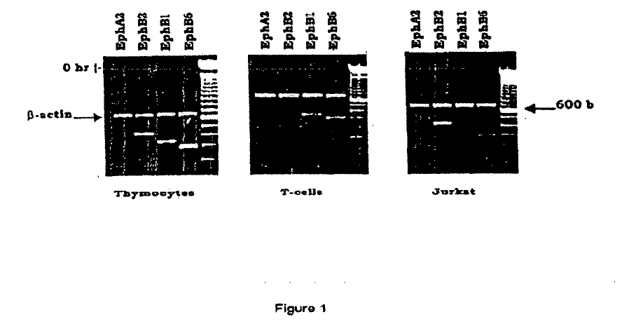

In the present invention it is demonstrated that Eph receptors such as EphB1, EphB2 and EphB6 are expressed on T lymphocytes (FIG. 1) and that modulating ephrins or the Eph receptor can be used to modulate an immune response such as a T cell response.

The present invention provides a method of modulating an immune response comprising administering an effective amount of a substance that modulates an ephrin or an Eph receptor to a cell or animal in need thereof. It is to be understood that the substance can act on either the ephrin and/or Eph receptor and the ephrin or Eph receptor may be present on an immune cell and/or on another cell that functionally associates with an immune cell.

In one embodiment, Eph receptors and their ephrin ligands modulate T cell or progenitor T cell responses such as T cell:cell adhesion or adhesion. The ephrin mediated modulation of T cell adhesion can be mediated by leucocyte focal adhesion-1 molecule (LFA-1). It is also shown that the EphB1 receptor enhances ephrin induced T lymphocyte adhesion while the EphB6 receptor likely antagonizes T lymphocyte adhesion.

The present invention also provides, in another embodiment, a method of modulating a T cell response comprising administering an effective amount of a substance that modulates an ephrin or an Eph receptor to a cell or animal in need thereof.

In one embodiment, the present invention provides a method of modulating T cell adhesion comprising administering an effective amount of a substance that modulates an ephrin or an EphB1 receptor to a cell or animal in need thereof.

In one embodiment, the present invention provides a method of inducing T cell adhesion comprising administering an effective amount of a substance that activates an EphB1 receptor to a cell or animal in need thereof.

In another embodiment, the present invention provides a method of inhibiting T cell adhesion comprising administering an effective amount of a substance that inhibits an EphB1 receptor to a cell or animal in need thereof.

In another embodiment, the present invention provides a method of inducing T cell adhesion comprising administering an effective amount of a substance that inhibits an EphB6 receptor to a cell or animal in need thereof.

In another embodiment, the present invention provides a method of preventing or inhibiting T cell adhesion comprising administering an effective amount of a substance that activates an EphB6 receptor to a cell or animal in need thereof.

In a further embodiment, the present invention provides a method of modulating chemotaxis and/or migration comprising administering an effective amount of a substance that modulates an ephrin or an Eph receptor to a cell or animal in need thereof.

Modulating chemotaxis and/or cell migration may be useful in treating or preventing cancer, such as the metastasis or cancer cells.

The inventors have also demonstrated that ephrin-Eph receptor signaling modulates TCR/CD3 induced apoptosis in thymocytes.

Accordingly, the present invention also provides a method of modulating apoptosis comprising administering an effective amount of a substance that modulates an ephrin or an Eph receptor to a cell or animal in need thereof. In one embodiment, the animal has an autoimmune disease, an allergy, a graft, or is a transplant recipient.

Substances that modulate an ephrin or Eph receptor can be selected from any substance which is capable of modulating (including activating or inhibiting) an ephrin and/or Eph receptor on an immune cell or a cell or matrix that associates with an immune cell. Some substances of the present invention are outlined in greater detail in Section III below. Preferably, the substance is a substance that binds to an Eph receptor such as an ephrin. More preferably, the ephrin is a soluble monomeric or oligomerized ephrin such as ephrin-B1 which is a ligand for the EphB1, EphB2 and EphB6 receptors. To activate an Eph receptor the ephrin is preferably oligomeric. To inhibit an Eph receptor the ephrin is preferably monomeric.

The finding of the present invention that modulating Eph receptors and ephrins play a role in T cells regulation has important implications in the treatment of various conditions. In particular, substances that modulate an ephrin or an Eph receptor that result in the suppression or down regulation of an immune response, such as a T cell response, can be useful in treating a wide variety of conditions wherein immune suppression is desired such as autoimmune disease, allergy, graft versus host disease, and transplantation. Immune suppression would also be desired in T cell cancers and lymphoid cancers.

In accordance with an aspect of the present invention, substances that modulate an ephrin or an Eph receptor that result in the activation, enhancement or up regulation of an immune response, such as a T cell response, can also be useful in treating a wide variety of conditions including most cancers and tumors (in vivo, ex vivo and/or in vitro).

Activation of self-reactive T cell clones by self antigens is a key event in the development of autoimmune disorders, while activation of T cells with foreign antigens initiates allergic reactions, graft rejection and transplant rejection. All of these processes require the proper adhesion of reactive T lymphocytes to the target cells, with subsequent initiation of TCR signaling and TCR-mediated responses. In one embodiment, the present invention demonstrates that the ephrin-B1 ligand and its EphB1 and EphB6 receptors can regulate the adhesion T cells. Therefore, inhibitory blocking monomeric forms of soluble ligands and receptors or stimulatory-oligomeric forms of soluble ligands and receptors, or antibodies to the ligands or receptors could be used to inhibit or promote cell-cell interaction and thus inhibit or slow down autoimmune disorders, allergic reactions or rejection processes.

Accordingly, the present invention provides a method of suppressing an immune response comprising administering an effective amount of a substance that modulates an ephrin or an Eph receptor to a cell or animal in need thereof. In one embodiment, the animal has an autoimmune disease, an allergy, a graft, or is a transplant recipient.

Accordingly, the present invention provides in one embodiment a method of activating, enhancing or up regulating an immune response comprising administering an effective amount of a substance that modulates an ephrin or an Eph receptor to a cell or animal in need thereof. For example, administration of an effective amount of the substance to an animal which has cancer or has a tumor against which an enhanced immune response, such as an enhanced T cell response, is desirable.

In accordance with an aspect of the present invention, substances that modulate an ephrin or Eph receptor that result in the enhancement or upregulation of an immune response, such as a T cell response, can be useful in treating disease wherein immune activation is desirable such as in the treatment of cancer.

The stimulation of immune reactions against tumor cells is a rapidly developing and viable form of anti-cancer therapy. The invention demonstrates in one embodiment that EphB receptors differently regulate T cell adhesion and responses. It is recognized in the present invention that this effect of the EphB receptors could be used to enhance the ability of T cells to recognize cancer cells. Employment of oligomeric or monomeric forms of soluble ligands could selectively activate those Eph receptors that are positive regulators of T cell adhesion, or inhibit negative regulators. In this manner, treatment with soluble ephrin proteins or derivatives, or analogues, could promote immune responses against tumor cells and/or cancer cells, leading to reduction or elimination of the tumor and/or cancer.

The metastatic activity of cancer cells depends upon their adhesive, migratory and chemotactic properties. The invention demonstrates in one embodiment that ephrin-B1 and its receptors EphB1 and EphB6 can regulate the adhesion of both normal and transformed T cells. Therefore, treatment of T cell malignancies with oligomeric or monomeric ligands could potentially inhibit or reduce the metastatic process. Modulation of integrin activation through regulation of Eph receptor activity, preferably using ephrin proteins, might be a significant method for regulation of invasive behaviour and consequently improve prognosis in malignancy.

All the known Eph receptors, except EphB6 (Gurniak and Berg. 1996), are catalytically active kinases, initiating phosphorylation cascades within the cell. Mutation and overexpression of receptor tyrosine kinases is often associated with tumorigenesis. Changes in expression and/or mutation of Eph receptors may contribute to the tumorigenesis of some types of cells of the immune system, by promotion of the migratory ability of these tumor cells, contributing to their tissue invasive or metastatic behaviour. Regulating the expression of these receptors, or regulating their activity by blocking, or stimulating them with soluble ephrin proteins or analogues in accordance with the present invention may provide a method for decreasing aggressive invasive or metastatic behaviour.

Accordingly, the present invention provides a method of including an immune response comprising administering an effective amount of a substance that modulates an ephrin or an Eph receptor to a cell or animal in need thereof. In one embodiment, the animal has cancer and the method reduces aggressive invasive or metastatic behaviour of the cancer,

III. Modulators of Ephrins or Eph Receptors

The substance which may be used according to the invention to modulate an ephrin or an Eph receptor can be any substance that is effective in modulating the activity of an immune cell and/or a cell that is functionally associated with the immune cell. The invention is not limited to a particular modulator. For example such substances may be selected from an oligomeric or monomeric soluble ephrins (such as ephrin-B1 or ephrin-B2) or Eph receptors (such as EphB1, EphB2 or EphB6), an antibody capable of binding an ephrin or an Eph receptor, an antisense molecule to an ephrin or an Eph receptor, a peptide mimetic based on an ephrin or Eph receptor or other substances identified in the screening assays described. Such substances may be readily available or may be prepared as hereinbelow described.

(a) Soluble Proteins

Soluble ephrin and Eph proteins represent a class of substances that may be used advantageously to modulate the activity of the ephrins and Eph receptors. Soluble proteins can be prepared by a number of conventional methodologies. GST fusion proteins of Eph receptor and ephrin extracellular domains, or activated or inactive variants thereof, can be created in the pGEX vector series (Pharmacia Biotech). When the vectors containing the cDNAs are transformed into bacteria by heat shock uptake, expression of the GST fusion proteins can be induced with 1 mM IPTG. After growth bacteria can be lysed by sonication and the addition of mild detergents. The resulting supernatant can be clarified by centrifugation and the released GST-fusion proteins purified by binding to Glutathione-Sepharose. After extensive washing these complexes can be checked for purity and quantitated by reference to standard proteins of similar molecular weight after staining with Coomassie Blue. Alternatively fusions of the Eph or ephrin proteins with MBP, His, ThioHis, Fc, Myc tag, HA tag, or other epitopes or domains may be used to allow other purification procedures to be utilized which may result in preferable activity of the purified protein. Fusion domains can be removed by the inclusion of a proteolytic cleavage site between the fusion partner and the ephrin or Eph protein.

(b) Antibodies

Antibodies represent a class of substances that may be used advantageously to modulate the activity of an ephrin or Eph receptor. Antibodies may be used to either inhibit, or stimulate the Eph receptor. Antibodies can be prepared which bind a distinct epitope in an unconserved region of the protein. An unconserved region of the protein is one that does not have substantial sequence homology to other proteins.

Conventional methods can be used to prepare the antibodies. For example, by using a peptide or fusion protein of an Eph receptor, polyclonal antisera or monoclonal antibodies can be made using standard methods. A mammal, (e.g., a mouse, hamster, or rabbit) can be immunized with an immunogenic form of the peptide which elicits an antibody response in the mammal. Techniques for conferring immunogenicity on a peptide include conjugation to carriers or other techniques well known in the art. For example, the protein or peptide can be administered in the presence of adjuvant. The progress of immunization can be monitored by detection of antibody titers in plasma or serum. Standard ELISA or other immunoassay procedures can be used with the immunogen as antigen to assess the levels of antibodies. Following immunization, antisera can be obtained and, if desired, polyclonal antibodies isolated from the sera.

To produce monoclonal antibodies, antibody producing cells (lymphocytes) can be harvested from an immunized animal and fused with myeloma cells by standard somatic cell fusion procedures thus immortalizing these cells and yielding hybridoma cells. Such techniques are well known in the art, (e.g., the hybridoma technique originally developed by Kohler and Milstein (Nature 256, 495-497 (1975)) as well as other techniques such as the human B-cell hybridoma technique (Kozbor et al., Immunol. Today 4, 72 (1983)), the EBV-hybridoma technique to produce human monoclonal antibodies (Cole et al. Monoclonal Antibodies in Cancer Therapy (1985) Allen R. Bliss, Inc., pages 77-96), and screening of combinatorial antibody libraries (Huse et al., Science 246, 1275 (1989)). Hybridoma cells can be screened immunochemically for production of antibodies specifically reactive with the peptide and the monoclonal antibodies can be isolated.

The term “antibody” as used herein is intended to include fragments thereof which also specifically react with an ephrin or an Eph receptor, or peptide thereof. Antibodies can be fragmented using conventional techniques and the fragments screened for utility in the same manner as described above. For example, F(ab′)2 fragments can be generated by treating antibody with pepsin. The resulting F(ab′)2 fragment can be further enzymatically treated to produce Fab′ fragments.

Chimeric antibody derivatives, i.e., antibody molecules that combine a non-human animal variable region and a human constant region are also contemplated within the scope of the invention. Chimeric antibody molecules can include, for example, the antigen binding domain from an antibody of a mouse, rat, or other species, with human constant regions. Conventional methods may be used to make chimeric antibodies containing the immunoglobulin variable region which recognizes the gene product of ephrins or Eph receptors of the invention (See, for example, Morrison et al., Proc. Natl Acad. Sci. U.S.A. 81, 6851 (1985); Takeda et al., Nature 314, 452 (1985), Cabilly et al., U.S. Pat. No. 4,816,567; Boss et al., U.S. Pat. No. 4,816,397; Tanaguchi et al., European Patent Publication EP171496; European Patent Publication 0173494, United Kingdom patent GB 2177096B). It is expected that chimeric antibodies would be less immunogenic in a human subject than the corresponding non-chimeric antibody.

Monoclonal or chimeric antibodies specifically reactive with a protein of the invention as described herein can be further humanized by producing human constant region chimeras, in which parts of the variable regions, particularly the conserved framework regions of the antigen-binding domain, are of human origin and only the hypervariable regions are of non-human origin. Such immunoglobulin molecules may be made by techniques known in the art, (e.g., Teng et al., Proc. Natl. Acad. Sci, U.S.A., 80, 7308-7312 (1983); Kozbor et al., Immunology Today, 4, 7279 (1983); Olsson et al., Meth. Enzymol., 92, 3-16 (1982)), and PCT Publication WO92/06193 or EP 0239400). Humanized antibodies can also be commercially produced (Scotgen Limited, 2 Holly Road, Twickenham, Middlesex, Great Britain.)

Specific antibodies, or antibody fragments, reactive against ephrins or Eph receptors proteins may also be generated by screening expression libraries encoding immunoglobulin genes, or portions thereof, expressed in bacteria with peptides produced from the nucleic acid molecules of the ephrins or Eph receptor. For example, complete Fab fragments, VH regions and FV regions can be expressed in bacteria using phage expression libraries (See for example Ward et al. Nature 341. 544-546: (1989); Huse et al., Science 246, 1275-1281 (1989); and McCafferty et al. Nature 348, 552-554 (1990)). Alternatively, a SCID-hu mouse, for example the model developed by Genpharm, can be used to produce antibodies or fragments thereof.

(c) Antisense Oligonucleotides

Antisense oligonucleotides that are complementary to a nucleic acid sequence from an ephrin or Eph receptor can also be used in the methods of the present invention to modulate the ephrins or Eph receptors.

The term “antisense oligonucleotide” as used herein means a nucleotide sequence that is complementary to its target.

The term “oligonucleotide” refers to an oligomer or polymer of nucleotide or nucleoside monomers consisting of naturally occurring bases, sugars, and intersugar (backbone) linkages. The term also includes modified or substituted oligomers comprising non-naturally occurring monomers or portions thereof, which function similarly. Such modified or substituted oligonucleotides may be preferred over naturally occurring forms because of properties such as enhanced cellular uptake, or increased stability in the presence of nucleases. The term also includes chimeric oligonucleotides which contain two or more chemically distinct regions. For example, chimeric oligonucleotides may contain at least one region of modified nucleotides that confer beneficial properties (e.g. increased nuclease resistance, increased uptake into cells),or two or more oligonucleotides of the invention may be joined to form a chimeric oligonucleotide.

The antisense oligonucleotides of the present invention may be ribonucleic or deoxyribonucleic acids and may contain naturally occurring bases including adenine, guanine, cytosine, thymidine and uracil. The oligonucleotides may also contain modified bases such as xanthine, hypoxanthine, 2-aminoadenine, 6-methyl, 2-propyl and other alkyl adenines, 5-halo uracil, 5-halo cytosine, 6-aza uracil, 6-aza cytosine and 6-aza thymine, pseudo uracil, 4-thiouracil, 8-halo adenine, 8-aminoadenine, 8-thiol adenine, 8-thiolalkyl adenines, 8-hydroxyl adenine and other 8-substituted adenines, 8-halo guanines, 8-amino guanine, 8-thiol guanine, 8-thiolalkyl guanines, 8-hydroxyl guanine and other 8-substituted guanines, other aza and deaza uracils, thymidines, cytosines, adenines, or guanines, 5-trifluoromethyl uracil and 5-trifluoro cytosine.

Other antisense oligonucleotides of the invention may contain modified phosphorous, oxygen heteroatoms in the phosphate backbone, short chain alkyl or cycloalkyl intersugar linkages or short chain heteroatomic or heterocyclic intersugar linkages. For example, the antisense oligonucleotides may contain phosphorothioates, phosphotriesters, methyl phosphonates, and phosphorodithioates. In an embodiment of the invention there are phosphorothioate bonds links between the four to six 3′-terminus bases. In another embodiment phosphorothioate bonds link all the nucleotides.

The antisense oligonucleotides of the invention may also comprise nucleotide analogs that may be better suited as therapeutic or experimental reagents. An example of an oligonucleotide analogue is a peptide nucleic acid (PNA) wherein the deoxyribose (or ribose) phosphate backbone in the DNA (or RNA), is replaced with a polyamide backbone which is similar to that found in peptides (P. E. Nielsen, et al Science 1991, 254, 1497). PNA analogues have been shown to be resistant to degradation by enzymes and to have extended lives in vivo and in vitro. PNAs also bind more strongly to a complementary DNA sequence due to the lack of charge repulsion between the PNA strand and the DNA strand. Other oligonucleotides may contain nucleotides containing polymer backbones, cyclic backbones, or acyclic backbones. For example, the nucleotides may have morpholino backbone structures (U.S. Pat. No. 5,034,506). Oligonucleotides may also contain groups such as reporter groups, a group for improving the pharmacokinetic properties of an oligonucleotide, or a group for improving the pharmacodynamic properties of an antisense oligonucleotide. Antisense oligonucleotides may also have sugar mimetics.

The antisense nucleic acid molecules may be constructed using chemical synthesis and enzymatic ligation reactions using procedures known in the art. The antisense nucleic acid molecules of the invention or a fragment thereof, may be chemically synthesized using naturally occurring nucleotides or variously modified nucleotides designed to increase the biological stability of the molecules or to increase the physical stability of the duplex formed with mRNA or the native gene e.g. phosphorothioate derivatives and acridine substituted nucleotides. The antisense sequences may be produced biologically using an expression vector introduced into cells in the form of a recombinant plasmid, phagemid or attenuated virus in which antisense sequences are produced under the control of a high efficiency regulatory region, the activity of which may be determined by the cell type into which the vector is introduced.

The antisense oligonucleotides may be introduced into tissues or cells using techniques in the art including vectors (retroviral vectors, adenoviral vectors and DNA virus vectors) or physical techniques such as microinjection. The antisense oligonucleotides may be directly administered in vivo or may be used to transfect cells in vitro which are then administered in vivo. In one embodiment, the antisense oligonucleotide may be delivered to macrophages and/or endothelial cells in a liposome formulation.

(d) Peptide Mimetics

The present invention also includes peptide mimetics of the ephrin or Eph receptor of the invention. For example, a peptide derived from a binding domain of an ephrin or Eph protein will interact directly or indirectly with an associated molecule in such a way as to mimic the native binding domain. Such peptides may include competitive inhibitors, enhancers, peptide mimetics, and the like. All of these peptides as well as molecules substantially homologous, complementary or otherwise functionally or structurally equivalent to these peptides may be used for purposes of the present invention.

“Peptide mimetics” are structures which serve as substitutes for peptides in interactions between molecules (See Morgan et al (1989), Ann. Reports Med. Chem. 24:243-252 for a review). Peptide mimetics include synthetic structures which may or may not contain amino acids and/or peptide bonds but retain the structural and functional features of a peptide, or enhancer or inhibitor of the invention. Peptide mimetics also include peptoids, oligopeptoids (Simon et al (1972) Proc. Natl. Acad. Sci USA 89:9367); and peptide libraries containing peptides of a designed length representing all possible sequences of amino acids corresponding to a peptide of the invention.

Peptide mimetics may be designed based on information obtained by systematic replacement of L-amino acids by D-amino acids, replacement of side chains with groups having different electronic properties, and by systematic replacement of peptide bonds with amide bond replacements. Local conformational constraints can also be introduced to determine conformational requirements for activity of a candidate peptide mimetic. The mimetics may include isosteric amide bonds, or D-amino acids to stabilize or promote reverse turn conformations and to help stabilize the molecule. Cyclic amino acid analogues may be used to constrain amino acid residues to particular conformational states. The mimetics can also include mimics of inhibitor peptide secondary structures. These structures can model the 3-dimensional orientation of amino acid residues into the known secondary conformations of proteins. Peptoids may also be used which are oligomers of N-substituted amino acids and can be used as motifs for the generation of chemically diverse libraries of novel molecules.

Peptides of the invention may also be used to identify lead compounds for drug development. The structure of the peptides described herein can be readily determined by a number of methods such as NMR and X-ray crystallography. A comparison of the structures of peptides similar in sequence, but differing in the biological activities they elicit in target molecules can provide information about the structure-activity relationship of the target. Information obtained from the examination of structure-activity relationships can be used to design either modified peptides, or other small molecules or lead compounds which can be tested for predicted properties as related to the target molecule. The activity of the lead compounds can be evaluated using assays similar to those described herein.

Information about structure-activity relationships may also be obtained from co-crystallization studies. In these studies, a peptide with a desired activity is crystallized in association with a target molecule, and the X-ray structure of the complex is determined. The structure can then be compared to the structure of the target molecule in its native state, and information from such a comparison may be used to design compounds expected to possess properties similar to those of the peptide having the desired activity.

(e) Ephrin or Eph Receptor Fragments, Analogs and Derivatives

The present invention extends to use of fragments, analogs and derivatives of ephrins and Eph receptors. Thus, for instance proteins or polypeptides which include one or more additions, deletions, substitutions or the like are encompassed by the present invention.

As used herein, “fragments”, “analogs” or “derivatives” of the polypeptides of the invention include those polypeptides in which one or more of the amino acid residues are substituted with a conserved or non-conserved amino acid residue (preferably conserved) and which may be natural or unnatural. In one embodiment, derivatives and analogs of polypeptides of the invention will have about 80% identity with the sequences of the exemplified ephrins or Eph receptors. That is, 80% of the residues are the same. In a further embodiment, polypeptides will have greater than 80% identity. In a further embodiment, polypeptides will have greater than 85% identity. In a further embodiment, polypeptides will have greater than 90% identity. In a further embodiment, polypeptides will have greater than 95% identity. In a further embodiment, polypeptides will have greater than 99% identity. In a further embodiment, analogs of polypeptides of the invention will have fewer than about 20 amino acid residue substitutions, modifications or deletions and more preferably less than 10.

These substitutions are those having a minimal influence on the secondary structure and hydropathic nature of the polypeptide. Preferred substitutions are those known in the art as conserved, i.e. the substituted residues share physical or Chemical properties such as hydrophobicity, size, charge or functional groups. These include substitutions such as those described by Dayhoff, M. in Atlas of Protein Sequence and Structure 5, 1978 and by Argos, P. In EMBO J. 8, 779-785, 1989. For example, amino acids, either natural or unnatural, belonging to one of the following groups represent conservative changes:

ala, pro, gly, gin, asn, ser, thr, val;

cys, ser, tyr, thr,

val, ile, leu, met, ala, phe;

lys, arg, orn, his:

and phe, tyr, trp, his.

The preferred substitutions also include substitutions of D-enantiomers for the corresponding L-amino acids.

One can use a program such as the CLUSTAL™ program to compare amino acid sequences. This program compares amino acid sequences and finds the optimal alignment by inserting spaces in either sequence as appropriate. It is possible to calculate amino acid identity or homology for an optimal alignment. A program like BLASTx™ will align the longest stretch of similar sequences and assign a value to the fit. It is thus possible to obtain a comparison where several regions of similarity are found, each having a different score. Both types of identity analysis are contemplated in the present invention.

Particularly preferred for comparing two polypeptide sequences is the BLAST 2 Sequences tool provided by the National Center for Biotechnology Information (NCBI), and which is available from NCBI in Bethesda, Md., or on the Internet at http://www.ncbl.nim.nih.gov/BLAST/. For a pairwise comparison: of two polypeptide sequences, the “BLAST 2 Sequences” tool Version 2.0.12 can be used with blastp set at the following default parameters: Matrix: BLOSUM62: Open Gap—11 and Extension Gap—1 penalties; Gap x drop-off—50; Expect—10; Word Size—3; Filter—on.

Also included are polypeptides which have fused thereto other compounds which alter the polypeptides biological or pharmacological properties e.g. polyethylene glycol (PEG) to increase half-life; leader or secretory amino acid sequences for ease of purification; prepro- and pro-sequences; and (poly)saccharides. Moreover, the polypeptides of the present invention can be modified by terminal —NH2 acylation (eg. by acetylation, or thioglycolic acid amidation, terminal carboxy amidation, e.g. with ammonia or methylamine) to provide stability, increased hydrophobicity for linking or binding to a support or other molecule.

(f) Other Modulators

In addition to soluble proteins, antibodies, antisense oligonucleotides and peptide mimetics, other substances that modulate ephrins or Eph receptors may also be identified. For example, substances that affect ephrins or Eph receptor activity can be identified based on their ability to bind to the ephrin or Eph receptor. Additional useful substances in the context of the present invention include, without limitation, non-proteinaceous compounds capable of binding to and activating or inhibiting an ephrin or an Eph receptor.

Substances which can bind with the ephrin or Eph receptor of the invention may be identified by reacting the ephrin or Eph receptor with a substance which potentially binds to the ephrin or Eph receptor, and assaying for complexes, for free substance, or for non-complexed ephrin or Eph receptor, or for activation of the ephrin or Eph receptor. In particular, a yeast two hybrid assay system may be used to identify proteins which interact with the EphB6 receptor (Fields, S. and Song, O., 1989, Nature, 340:245-247) or a ligand binding or ligand replacement assay system (Blechman, J. M. et al. (1993); Blechman, J. M. et al. (1995); Lev et al. (1993)). Systems of analysis which also may be used include ELISA, BIAcore (Bartley, T. D., at al. (1994)).

As an example, a protein ligand for the Eph receptors can be isolated by using the extracellular domain of the receptor as an affinity reagent. Concentrated cell culture supernatants can be screened for receptor binding activity using immobilized receptor in a surface plasmon resonance detection system (BIAcore). Supernatants from selected cell lines can then be fractionated directly by receptor affinity chromatography.

Conditions which permit the formation of substance and ephrin or Eph receptor complexes may be selected having regard to factors such as the nature and amounts of the substance and the protein.

The substance-protein complex, free substance or non-complexed protein may be isolated by conventional isolation technique, for example, salting out, chromatography, electrophoresis, gel filtration, fractionation, absorption, polyacrylamide gel electrophoresis, agglutination, or combinations thereof. To facilitate the assay of the components, antibody against the ephrin or Eph receptor or the substance, or labelled ephrin or Eph receptor, or a labelled substance may be utilized. The antibodies, proteins, or substances may be labelled with a detectable substance as described above.

The ephrin or Eph receptor, or the substance used in the method of the invention may be insolubilized. For example, the ephrin or Eph receptor or substance may be bound to a suitable carrier. Examples of suitable carriers are agarose, cellulose, dextran, Sephadex, Sepharose, carboxymethyl cellulose polystyrene, filter paper, ion-exchange resin, plastic film, plastic tube, glass beads, polyamine-methyl vinyl-ether-maleic acid copolymer, amino acid copolymer, ethylene-maleic acid copolymer, nylon, silk, etc. The carrier may be in the shape of, for example, a tube, test plate, beads, disc, sphere etc.

The insolubilized protein or substance may be prepared by reacting the material with a suitable insoluble carrier using known chemical or physical methods, for example, cyanogen bromide coupling.

The proteins or substance may also be expressed on the surface of a cell.

The invention also contemplates a method for assaying for an agonist or antagonist of the ephrin or Eph receptor. The agonist or antagonist may be an endogenous physiological substance or it may be a natural or synthetic substance. Substances that are capable of binding the ephrin or Eph receptor may be identified using the methods set forth herein.

It will be understood that the agonists and antagonists that can be assayed using the methods of the invention may act on one or more of the binding sites on the protein or substance including agonist binding sites, competitive antagonist binding sites, non-competitive antagonist binding sites or allosteric sites.

The invention also makes it possible to screen for antagonists that inhibit the effects of an agonist of the ephrin or Eph receptor or its active partners. Thus, the invention may be used to assay for a substance that competes for the same binding site of the ephrin or Eph receptor or its active partners.

IV. Pharmaceutical Compositions

The above described substances that modulate an ephrin or Eph receptor may be formulated into pharmaceutical compositions for administration to subjects in a biologically compatible form suitable for administration in vivo. By “biologically compatible form suitable for administration in vivo” is meant a form of the substance to be administered in which any toxic effects are outweighed by the therapeutic effects. The substances may be administered to living organisms including humans, and animals.

Accordingly, the present invention provides a composition comprising an effective amount of a substance that modulates an ephrin or Eph receptor in admixture with a suitable diluent or carrier. Such compositions are useful in the therapeutic methods described above.

Administration of an effective amount of pharmaceutical compositions of the present invention is defined as an amount effective, at dosages and for periods of time necessary to achieve the desired result. For example, an effective amount of a substance may vary according to factors such as the disease state, age, sex, and weight of the individual, and the ability of the substance to elicit a desired response in the individual. Dosage regima may be adjusted to provide the optimum therapeutic response. For example, several divided doses may be administered daily or the dose may be proportionally reduced as indicated by the exigencies of the therapeutic situation.

An active substance may be administered in a convenient manner such as by injection (subcutaneous, intravenous, etc.), oral administration, inhalation, transdermal application, or rectal administration. Depending on the route of administration, the active substance may be coated in a material to protect the compound from the action of enzymes, acids and other natural conditions which may inactivate the compound. If the active substance is a nucleic acid encoding, for example, a modified Eph receptor it may be delivered using techniques known in the art.

The compositions described herein can be prepared by per se known methods for the preparation of pharmaceutically acceptable compositions which can be administered to subjects, such that an effective quantity of the active substance is combined in a mixture with a pharmaceutically acceptable vehicle. Suitable vehicles are described, for example, in Remington's Pharmaceutical Sciences (Remington's Pharmaceutical Sciences, Mack Publishing Company, Easton, Pa., USA 1985) or Handbook of Pharmaceutical Additives (compiled by Michael and Irene Ash, Gower Publishing Limited, Aldershot, England (1995)). On this basis, the compositions include, albeit not exclusively, solutions of the substances in association with one or more pharmaceutically acceptable vehicles or diluents, and may be contained in buffered solutions with a suitable pH and/or be iso-osmotic with physiological fluids. In this regard, reference can be made to U.S. Pat. No. 5,843,456. As will also be appreciated by those skilled, administration of substances described herein may be by an inactive viral carrier.

The following non-limiting examples are illustrative of the present invention:

### EXAMPLES

### Example 1

### Regulation of Lymphocytes by Ephrin Stimulation

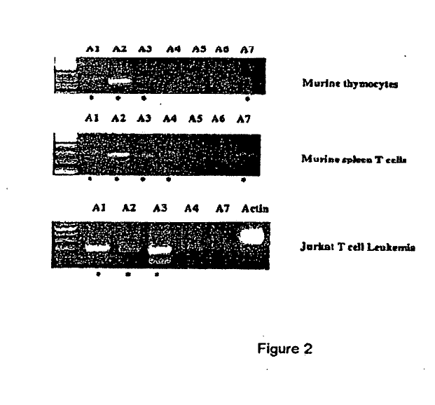

The present experiments with cells from the immune system demonstrate that treatment of primary or transformed human T cells with soluble oligomerized ephrin-B1 (a ligand for the EphB1, -B2 and -B6 receptors) induces the formation of cell-cell contacts (FIGS. 2A&B).

Cell-cell adhesion in T lymphocytes is known to be controlled, in particular by β2-integrin receptors and their interaction with the ligands ICAM-1, ICAM-2 and ICAM-3. The most important of these receptors on T cells appears to be the leukocyte focal adhesion-1 molecule (LFA-1). LFA-1 is composed of two protein chains, CD11a and CD18, and is involved in cell adhesion events through binding to ICAM-1, ICAM-2 and ICAM-3 expressed on the surface of neighbouring cells. On naïve T cells, these receptors do not demonstrate high affinity ligand binding and the cells require stimulation, for example, with chemokines, for efficient ligand binding to occur and subsequent promotion of cell-cell adhesion.

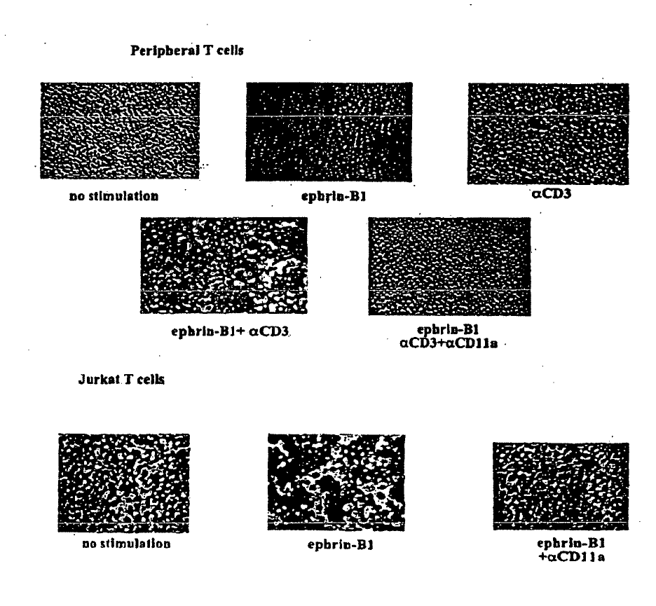

Ephrin-B1-induced clustering of T cells can be strongly and specifically inhibited by the presence of a blocking antibody to LFA-1 (FIG. 4). This suggests that ephrin-B1-induced T lymphocyte cell-cell adhesion is mediated by LFA-1.

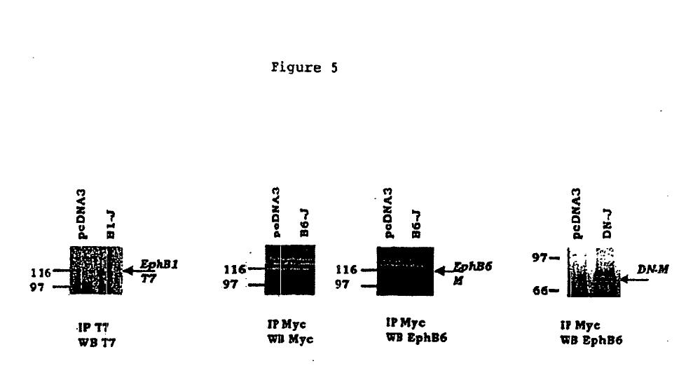

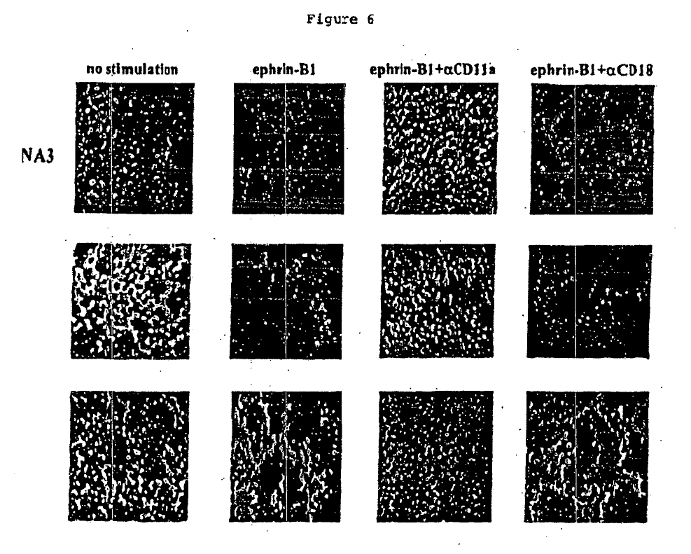

To determine which of the EphB receptors could specifically mediate ephrin-B1-induced T lymphocyte cell-cell adhesion, the inventors generated stable overexpression of EphB1 (B1-J), EphB6 (B6-J) and a dominant negative EphB6 mutant (DN-J,—intracellular domain deleted) in human T cells Jurkat (FIG. 5). Overexpression of the EphB6 receptor in Jurkat T cells did not dramatically affect ephrin-B1-induced cell-cell adhesion, although in the majority of the experiments it partially attenuated the effect of ephrin-B1 stimulation (not shown). The low and variable effect of EphB6 overexpression could be due to a high level of endogenous receptor expression. Indeed, expression of a dominant negative mutant of EphB6 resulted in a strong and consistent enhancement of both spontaneous and ephrin-B1-induced cell-cell adhesion; presumably due to the elimination of a negative signal from the endogenous EphB6 receptor (FIG. 6). Overexpression of the EphB1 receptor also strongly enhanced the effect of ephrin-B1 treatment, as well as increasing spontaneous T cell adhesion (FIG. 6). In each case, increased adhesion responses could be specifically inhibited by the presence of a blocking antibody to LFA-1, but not by a non-blocking control anti-LFA-1 anti-sera. Together, these findings demonstrate that the EphB1 and EphB6 receptors both regulate T cell adhesion, although in antagonistic manner. These experiments demonstrate that in particular, the EphB1 receptor is capable of mediating ephrin-B1-induced T lymphocyte adhesion, while an EphB6-generated signal is likely to antagonize it.

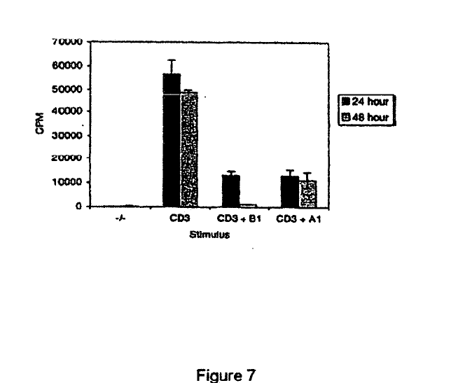

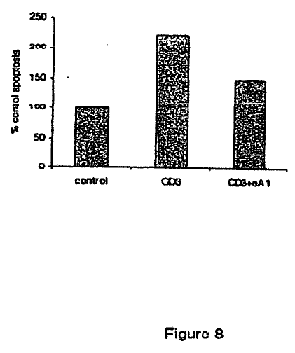

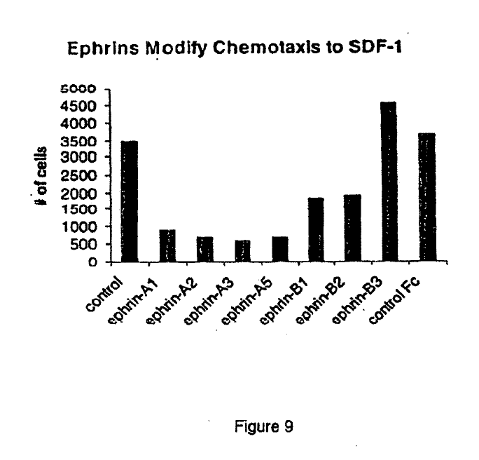

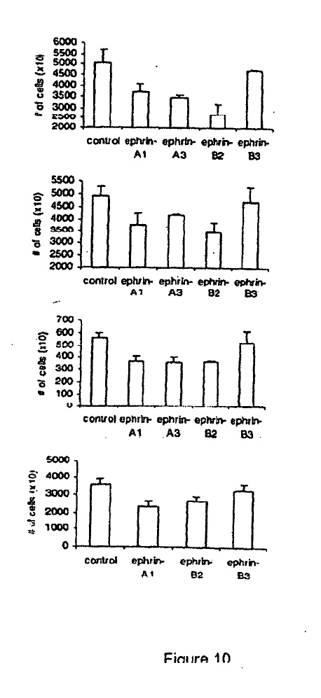

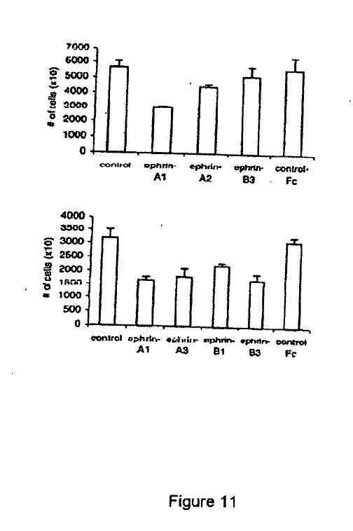

Thus, the inventors demonstrate for the first time that Eph receptors can control cell adhesion in T lymphocytes. The formation of cell-cell and cell-matrix contacts are key events in initiation and regulation of the majority of T cell responses, including T cell homing, targeting, interaction with antigen presenting cell, TCR-antigen interaction and TCR-signaling. As a result T cell adhesion modulates T cell-mediated immune responses and controls T cell differentiation, migration, proliferation, survival and activation induced cell death. Indeed experiments performed by inventors indicate that ephrin-Eph signaling modulates TCR/CD3 induced proliferation (FIG. 7) and apoptosis in T lymphocytes and thymocytes, potentially affecting mature T cell activation and negative selection of self-reacting T cells (FIG. 8), and affects chemotactic-induced T cell said thymocyte migration (FIGS. 9, 10 and 11). The influence of ephrin co-stimulation upon the migratory response of the Jurkat T cell line, human peripheral T lymphocytes and thymocytes to the chemotactic factor SDF-1alpha was examined in vitro. Significant reductions in T cell chemotaxis were observed with all four members of the ephrin-A subfamily. Ephrin-B1 and -B2 were also found to exhibit some inhibitory effect. A control Fc fusion protein had no effect. Thus a general property of ephrins would appear to be the ability to modify T cell chemotaxis, presumably through modulation of cytoskeletal structure. Correct targeting through appropriate responses to attractant chemotactic factors plays an important role in directing T cells to the correct subcompartments of lymphoid tissues and in targeting to protective surfaces throughout the body. The combined effect of targeting through ephrins and chemotactic factors would permit precise control over T cell movement and compartmentalization. Inappropriate targeting through dysregulation of chemoattractant receptors may play an important role in the spread of cancer cells throughout the body.

Both the sets of experiments examining T cell adhesion and examining chemotactic responses, revealed that T cell responses are modulated by a wide range of ephrins, not limited to either the A or B subfamily, and that within these subfamilies there is a considerable degree of specificity. In some cases, e.g. in chemotaxis, a number of the ephrins cause the same effect, the ephrin-A subfamily, presumably due to utilization of the same Eph receptor(s), while in others the ephrin-B subfamily, distinct responses to stimulation with different ephrin-B ligands would suggest distinct EphB receptor usage.

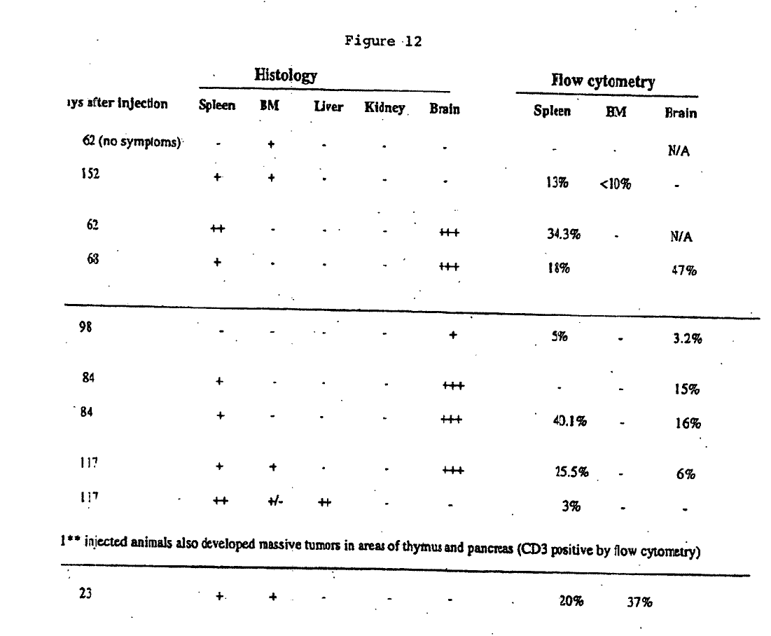

Combining the observations regarding the effects of Eph receptor usage activation on T cell adhesion and migration, the inventors examined the ability of altered Eph receptor expression or function to change T leukemia cell aggressiveness and tissue targeting in vivo (FIG. 12). T cells with altered Eph receptor expression were injected into mice. When the mice became sick, they were sacrificed arm analysed for the presence of the human T cells, by staining of tissue sections and flow cytometry analysis of single cell suspensions of organs. These experiments clearly demonstrated that alteration of Eph expression or function by expressing a partially-functional interfering EphB1 mutant (EphB1**) or a dominant negative form of EphB6-cytoplasmic domain deleted-(EphB6 DN), dramatically changed the aggressiveness the leukemia cells. Mice injected with cells bearing these mutated receptors fell sick significantly before control animals injected with the original unmodified T leukemia cells. When these control animals were sacrificed at the same time, few T leukemia cells were detectable in their tissues. Mice injected with cells bearing the mutated EphB6 and EphB1 receptors were found to have massive infiltration of the brain with the T cells. Such findings were not observed in control animals where infiltration appeared to occur primarily in the bone marrow and spleen. Mice injected with EphB1 mutant cells also developed large thymic and pancreatic tumour masses not observed in the control animals. The control animal which died in experiment number 2 did not demonstrate significant infiltration and appeared to have died from non-cancer related causes. Thus the alterations in T cell adhesion and migration observed in in vitro assays appear to translate into significantly altered behaviour in vivo. Modulation of Eph receptor function would therefore appear to provide a method for modifying the behaviour of cancer cell aggressiveness and tissue targeting, and is also likely to extend to modifying normal T cell tissue invasiveness and targeting.

In sum accumulated data strongly suggest that Eph receptors and their ligands (ephrins) are powerful regulators of various aspects of T cell differentiation, behaviour, and T cell mediated immune response.

### Example 2

### Modulation of T Cell Adhesion Through Ephrins and Ephs

As the co-ordination of multiple receptor contacts, including the integrins, is required for the correct recognition of antigen presenting cells by T lymphocytes, stimulation of the appropriate Eph receptor, or receptor combination, and lack of inappropriate stimulation may be required for productive interaction between immunological relevant cells. The investigators experiments reveal data consistent with Eph mediated alterations in cell-cell interaction, through integrin modulation.

Due to the membrane bound nature of both the Eph receptor and ephrin ligand, an important feature of receptor-ligand interaction is the necessity for the formation of cell-cell contact. Activation of the TCR complex occurs in an area of T-ell contact with an antigen-presenting cell, and activated TCR complexes may therefore come into close proximity with EphB receptors. As TCR signaling responses are dependent upon re-organization of the actin cytoskeleton and signals transmitted via integrin receptors, both processes regulated by activated Eph receptors in neuronal and endothelial cells (Huynh-Doh et al 1999, Zisch and Pasquale 1997, Becker 2000), and as directly demonstrated by the investigators in lymphocytes, the potential for productive interaction between those receptor pathway is high.

Dynamic re-organization of the actin cytoskeleton is critical for many stages of T lymphocyte activation and function (Abraham et al., 1999; Ticchioni et al., 1993; Vivinus-Nebot et al., 1999; Wulfing and Davis, 1998). These include the formation of initial contact with antigen presenting cells, where physical tethering through integrins and TCR co-receptors activates actin reorganization and orientates the T call toward the site of contact, the formation of stable contact after initiation of TCR signaling to permit long term responses to develop, and the migration of T cells through tissues in response to chemoattractants.

Disruption of actin polymerization with cytochalasin D or Clostridium botulinum toxin inhibits T lymphocyte responses to antigen (Valitutti et al. 1995), demonstrating the importance of actin-reorganization to TCR signaling.

The area of stable contact formed between a T cell and APC displays significant structural organization, the receptors being tightly organized into what has been termed, by analogy with the neural system, an immunological synapse or SMAC (supramolecular activation cluster) (Dustin and Shaw. 1999, Grakoui et al. 1999). Although formation of this stable contact is not necessary to initiate signaling, it is required for effective T cell proliferation and differentiation. Assembly of the synapse and subsequent responses are strictly dependent upon cell adhesion, integrin receptor signaling and actin cytoskeleton re-organization (Dustin and Chan, 2000). These responses cannot be mediated by the TCR, as changes in the actin cytoskeleton are detectable prior to antigen recognition, and must therefore be induced by signaling through co-receptors such as GD28, CD2 or LFA-1. In addition, engagement of co-receptors can serve to modify TCR signaling, enhancing or inhibiting responses; possibly in a manner related to cytoskeleton re-arrangement and consequently to TCR distribution. Although the dependence of antigen receptor activation upon integrin function has already been demonstrated, the mechanism regulating integrin activation remains unclear. Activation of T cell Eph receptors, through binding to APC expressed ephrins, may stimulate β2-integrins and increase their affinity for ligand. Integrin activation in turn can cause cytoskeleton reorganization conducive to the initiation, or inhibition, of TCR signaling. Alternatively, failure to correctly engage Eph receptors may result in a transient adhesion. Stimulation of the appropriate Eph receptor, or combination of receptors, and lack of inappropriate stimulation may therefore be required for productive interaction. In this manner, Ephs may regulate formation of cell-cell contact and act as co-receptors for TCR signaling.

### Example 3

### Cell Adhesion Assay

To examine EphB mediated changes in the activation of β1-integrin binding to extracellular matrix, ³⁵S-Methionine labeled cells can be adhered to fibronectin, soluble VCAM-1 or laminin coated wells. After washing, cells can be released with trypsin, lysed and analyzed by scintillation β-counting. Alternatively, bound cells will be fixed and quantitated by crystal violet staining. β2-integrin activation can be assessed by flow cytometry analysis of binding to ICAM1-3. Integrin blocking antibodies can be used to determine which β2-integrins are involved in ephrin-induced aggregation.

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

### FULL CITATIONS FOR REFERENCES REFERRED TO IN THE SPECIFICATION

Abraham, C., Griffith, J., and Miller, J. (1999). The dependence for leukocyte function-associated antigen-1/ICAM-1 interactions in T cell activation cannot be overcome by expression of high density TCR ligand. J Immunol 162, 4399-405.

Acuto, O. & Cantrell, D. T cell activation and the cytoskeleton. Annu Rev Immunol 18, 165 84 (2000).
Adams, R. H. Wilkinson, G. A., Weiss, C., Diella, F., Gale, N. W., Deutsch, U., Risau, W., and Klein, R. (1999). Roles of ephrinB ligands and EphB receptors in cardiovascular development: demarcation of arterial/venous domains, vascular morphogenesis, and sprouting angiogenesis, Genes Dev 13, 295-306.
Blecker, E., Huynh-Do, U., Holland, S., Pawson, T., Daniel, T. O., and Skolnik, E. Y. (2000). Nck-interacting Ste20 kinase couples Eph receptors to c-Jun N-terminal kinase and integrin activation. Mol Cell Biol 20, 1537-45.
Berclaz, G., et al., Expression of the receptor protein tyrosine kinase myk-1/htk in normal and malignant mammary epithelium. Biochem Biophys Res Commun. 1996. 226(3): p. 869-75.
Clossek, T., and Ullrich, A. (1997). Identification of Elf-1 and B61 as high affinity ligands for the receptor tyrosine kinase MDK1. Oncogene 14, 35-43.
Daniel, T. O., Stain, C., Cerratti, D. P., St, J. P., Robert, B., and Abrahamson, D. R. (1996). ELK and LERK-2 in developing kidney and microvascular endothelial assembly. Kidney International Supplement.
Davis, S., Gale, N. W., Aldrich, T. H., Maisonpierre, P. C., Lhotak, V., Pawson, T., Goldfarb, M., and Yancopoulos, G. D. (1994). Ligands for EPH-related receptor tyrosine kinases that require membrane attachment or clustering for activity. Science 266, 816-9.
Drescher, U., Kremoser, C. Handwerker, C., Loschinger, J. Noda. M., and Bonhoeffer, F. (1995). In vitro guidance of retinal ganglion cell axons by RAGS, a 25 kDa tectal protein related to ligands for Eph receptor tyrosine kinases. Cell 82, 359-70.
Dustin, M. L. & Shaw, A. S. Costimulation: building an immunological synapse. Science 283, 649-50 (1999).
Dustin, M. L., and Chan, A. C. (2000). Signaling takes shape in the immune system. Cell 103, 283-94.
Flanagan, J. G. and Vanderhaeghen, P. (1998). The ephrins and Eph receptors in neural development. Annu Rev Neurosci 21, 309-45.
Gao, P. P., Yue, Y., Cerretti, D. P., Dreyfus, C., and Zhou, R. (1999). Ephrin-dependent growth and pruning of hippocampal axons. Proc Natl Acad Sci USA 96, 4073-7.
Grakoui, A. et al. The immunological synapse: a molecular machine controlling T cell activation [see comments]. Science 285, 221-7 (1999).
Gumiak, C. B., and Berg, L. J. (1996). A new member of the Eph family of receptors that lacks protein tyrosine kinase activity. Oncogene 13, 777-86.
Holsinger, L. J. Graef, I. A. Swat. W. Chi, T. Bautista, D. M., Davidson, L., Lewis, R. S., Alt, F. W., and Crabtree, G. R. (1998). Defects in actin-cap formation in Vav-deficient mice implicate an actin requirement for lymphocyte signal transduction. Curr Biol 8, 563-72.
Hornberger, M. R., Dutting, D., Clossek, T., Yamada, T., Handwerker, c., Lang, S., Weth, F., Huf, J., Wessel, R., Logan, C., Tanaka, H., and Drescher, U. (1999). Modulation of EphA receptor function by coexpressed ephrinA ligands on retinal ganglion cell axons. Neuron 22, 731-42.
Hsueh, Y. P., and Sheng, M. (1998). Eph receptors, ephrins, and PDZs gather in neuronal synapses [comment]. Neuron 21, 1227-9.
Huynh-Do, U., Stein, E., Lane, A. A., Liu, H., Cerretti, D. P., and Daniel. T. O. (1999). Surface densities of ephrin-B1 determine EphB1-coupled activation of cell attachment through alphavbeta3 and alpha5beta1 integrins. Embo J 18, 2165-73.
Kiyokawa, E., et al., Overexpression of ERK, an EPH family receptor protein tyrosine kinase, in various human tumors. Cancer Res. 1994. 54(14): p. 3645-50.
Krull, C. E., Lansford, R., Gale, N. W., Collazo, A. Marcelle, C., Yancopoulos, G. D., Fraser, S. E., and Bronner, F. M. (1997). Interactions of Eph-related receptors and ligands confer rostrocaudal pattern to trunk neural crest migration. Current Biology 7, 571-80.
Maru, Y., H. Hirai, and F. Takaku, Overexpression confers an oncogenic potential upon the eph gene. Oncogene, 1990. 5(3): p. 445-7.
Matsuoka, H., Iwata, N., Ito, M., Shimoyama, M., Nagata, A., Chihara, K., Takai, S., and Matsui, T. (1997) Expression of a kinase-defective Eph-like receptor in the normal human brain. Biochemical & Biophysical Research Communications 235, 487-92.
Melma, L., Kljavin, I. J., Moran, P., Shih, A, Winslow, J. W., and Caras, I. W. (1997). AL-1-induced growth cone collapse of rat cortical neurons is correlated with REK7 expression and rearrangement of the actin cytoskeleton. Eur J Neurosci 9, 177-88.
Melma, L., Moran, P., Matthews, W., and Caras, I. W. (1997a). Lerk2 (ephrin-B1) is a collapsing factor for a subset of cortical growth cones and acts by a mechanism different from AL-1 (ephrin-A5). Mol Cell Neurosci 9, 314-28.
O'Leary, D. D., and Wilkinson, D. G. (1999). Eph receptors and ephrins in neural development. Curr Opin Neurobiol 9, 65-73.
Pandey, A., Shao, H., Marks, R. M., Polverini, P. J., and Dixit, V. M. (1995). Role of B61, the ligand for the lack receptor tyrosine kinase, In TNF-alpha-induced angiogenesis. Science 268, 567-9.
Robinson, D., et al., A tyrosine kinase profile of prostate carcinoma. Proc Natl Acad Sci USA, 1996. 93(12): p. 5958-62.
Sakano, S., Serizawa, R., Inada, T., Iwama, A., Itoh, A., Kato, C., Shimizu, Y., Shinkai, F., Shimizu, R., Kondo, S. Ohno, M., and Suda, T. (1996). Characterization of a ligand for receptor protein-tyrosine kinase HTK expressed in immature hematopoietic cells. Oncogene 13, 813-22.
Snapper, S. B., Rosen, F. S., Mizoguchi, E., Cohen, P., Khan, W., Liu, C. H., Hagemann, T. L., Kwan, S. P., Ferrini, R., Davidson, L., Bhan, A. K., and Alt, F. W. (1998). Wiskott-Aldrich syndrome protein-deficient mice reveal a role for WASP in T but not B cell activation. Immunity 9, 81-91.
Sulman, E. P., et al., ECK, a human EPH-related gene, maps to 1p36.1, a common region of alteration in human cancers. Genomics, 1997. 40(2): p. 371-4.
Tang, X. X., et al., Coexpression of transcripts encoding EPHB receptor protein tyrosine kinases and their ephrin-B ligands in human small cell lung carcinoma. Clin Cancer Res, 1999. 5(2): p. 455-60.
Ticchioni, M., Aussel, C., Breittmayer, J. P., Manie, S., Pelassy, C., and Bernard, A. (1993). Suppressive effect of T cell proliferation via the CD29 molecules. The CD29 mAb 1 “K20” decreases diacylglycerol and phosphatidic acid levels in activated T cells. J Immunol 151, 119-27.
Valitutti, S., Dessing, M., Aktories, K., Gallati, H., and Lanzavecchia, A. (1995). Sustained signaling leading to T cell activation results from prolonged T cell receptor occupancy. Role of T cell actin cytoskeleton. J Exp Med 181, 577-84.
Vivinus-Nebot, M. Ticchioni, M., Mary, F., Hofman, P. Quaranta, V., Rousselle, P., and Bernard, A. (1999). Laminin 5 in the human thymus: control of T cell proliferation via alpha6beta4 integrins. J Cell Biol 144, 553-74.
Vogl, T., et al. Overexpression of Lerk-5/EpIg5 messenger RNA: a novel marker for increased tumorigenicity and metastatic potential in human malignant melanomas. Clin Cancer Res, 1998. 4(3): p. 791-7.
Wang, H. U., Chen, Z. F., and Anderson, D. J. (1998). Molecular distinction and angiogenic interaction between embryonic arteries and veins revealed by ephrin-B2 and its receptor Eph-B4. Cell 93, 741-53.
Wulfing, C., and Davis, M. M. (1998). A receptor/cytoskeletal movement triggered by costimulation during T cell activation. Science 282, 2266-9.
Wulfing, C., Sjaastad, M. D., and Davis, M. M. (1998). Visualizing the dynamics of T cell activation; intracelluar adhesion molecule 1 migrates rapidly to the T cell/B cell interface and acts to sustain calcium levels. Proc Natl Acad Sci USA 95, 6302-7.
Zhou, R. (1998). The Eph family receptors and ligands. Pharmacology & Therapeutics 77, 151-81.
Zisch, A. H., and Pasquale, E. B. (1997). The Eph family: a multitude of receptors that mediate cell recognition signals. Cell & Tissue Research 290, 217-26.

1.-20. (canceled)

21. A method for identifying a substance that modulates immune cell adhesion, comprising:
contacting an ephrin or an Eph receptor with a test substance; and
determining whether said ephrin or Eph receptor is modulated in the presence of said test substance, a modulation of said ephrin or Eph receptor being an indication that said test substance is useful for modulating immune cell adhesion.

22.-24. (canceled)

25. A method for identifying a substance useful for treating cancer through modulation of an adhesive, migratory or chemotactic property of a cancer cell, comprising:
contacting an ephrin or an Eph receptor with a test substance; and
determining whether said ephrin or Eph receptor is modulated in the presence of said test substance, a modulation of said ephrin or Eph receptor being an indication that said test substance is useful for treating cancer through modulation of an adhesive, migratory or chemotactic property of a cancer cell.
